(19) United States
(12) Patent Application Publication
(10) Pub. No.: US 2009/0163440 A1
(43) Pub. Date: Jun. 25, 2009

(54) Ion-Channel Regulator Compositions and Methods of Using Same

(76) Inventors: David D. Waddell, Shreveport, LA (US); Andrew A. Marino, Belcher, LA (US); Oleg V. Kolomytkin, Shreveport, LA (US)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 12/238,908

(22) Filed: Sep. 26, 2008

Related U.S. Application Data
(60) Provisional application No. 60/975,355, filed on Sep. 26, 2007.

(51) Int. Cl. A61K 31/715; A61K 31/4422; A61K 31/554; A61K 31/56; A61P 19/02

(52) U.S. Cl. 514/54; 514/356; 514/211.07; 514/171

(57) ABSTRACT

A composition and method for the treatment of degenerative joint disease is disclosed. The composition includes a combination of a first ion-channel regulator, at least a second ion-channel regulator, and a pharmaceutically acceptable carrier suitable for intraarticular injection. Methods and kits for treating a degenerative joint disease are also disclosed.

### RELATED APPLICATION

This application claims priority to U.S. Ser. No. 60/975,355 filed Sep. 26, 2007, hereby incorporated by reference in its entirety.

### FIELD OF INVENTION

The present invention relates in part to compositions that include two or more ion-channel regulators for the treatment of pain and inflammation in body tissues, including the synovial cells of body joints.

### BACKGROUND

Arthritis is degenerative joint disease characterized by inflammation of a joint and/or synovial tissue and membranes. There are many forms of arthritis, including osteoarthritis (hypertrophic or degenerative arthritis), rheumatoid arthritis, psoriatic arthritis, arthritis due to infection (tuberculosis, Lyme disease, rheumatic fever, etc.), suppurative arthritis, juvenile arthritis, and gouty arthritis.

Osteoarthritis is especially common among older people, and usually affects a joint on one side of the body. Cartilage and bone in the joint are primarily affected in osteoarthritis, and cartilage in the joint may break down and wear away, causing pain, swelling, and loss of motion of the joint. Rheumatoid arthritis is a systemic disease which, when manifested in joints, primarily affects the synovial membrane. Rheumatoid arthritis typically begins at a younger age than osteoarthritis, is usually present bilaterally in the joints, and sometimes results in feelings of sickness, tiredness, and fever.

The proteins known as cytokines are important factors in the onset and maintenance of inflammation. Cytokines, which are produced by synovial lining cells, cartilage cells, as well as by other types of cells, regulate numerous biological responses, including cell growth, and the nature and extent of proteins that are made by cells. Cytokines include interferons (IFNs), colony stimulating factors (CSFs), interleukins (ILs), and tumor necrosis factors (TNFs). The presence of inflammatory cytokines (IL-1, IL-8, TNF) initiates a series of complex cellular and molecular events, including the expression of adhesion molecules, the production of secondary inflammatory mediators (prostaglandins, leukotrienes), and the production of growth factors. Elevated tissue levels of IL-1, IL-8, and TNF are found in arthritis and in other inflammatory conditions.

In osteoarthritis, the cartilage that covers the ends of the bones that form the joint can be slowly degraded by the action of various enzymes, particularly the matrix metalloproteinases (MMPs) which are secreted into the synovial fluid of the joint by the synovial lining cells in response to stimulation by various pro-inflammatory cytokines, particularly IL-1 and TNF. The destruction of cartilage by the MMPs can perpetuate the inflammatory reaction and lead to joint pain associated with osteoarthritis.

Ion channels are glycoprotein structures located in the membrane of cells, including synovial cells and cartilage cells, which allow ions, particularly monovalent and divalent cations and anions, to pass through the membrane. Ion-channel regulators, typically chemical agents, may alter the entry of certain ions into or out of cells and cellular organelles, depending on whether the intracellular or extracellular concentration of the particular ion is greater, and on the electrical potential difference that exists between the inside and the outside of the cell. The combined effect of the concentration difference and the electrical potential difference is called the electrochemical gradient. When the gate of an ion channel is open, the ions will flow down their electrochemical gradient unless they are prevented from doing so as, for example, by means of a chemical ion-channel regulator. Ion-channel regulators are commonly used for treating a variety of conditions, including cardiac conditions such as atrial fibrillation, supraventricular tachycardias, hypertrophic cardiomyopathy and hypertension, as well as migraine headaches, the prevention of brain damage, and other disorders.

There is no known cure for e.g., osteoarthritis, and consequently clinical efforts aimed at treating it are presently directed toward symptomatic relief of pain. Joint replacement surgery may be advised in severe cases. Despite the availability of a wide range of medications and treatment modalities for arthritis and inflammatory diseases in general, none has proved to be entirely satisfactory for osteoarthritis. In particular, there remains a need for innovative treatments that target the underlying cause of osteoarthritis, and thereby help reduce, eliminate, or slow its progression (expressed symptomatically by bone erosion, cartilage erosion, inflammation, swelling, abnormal neovascularization, etc.)

### SUMMARY

According to its major aspects and broadly stated, the present invention provides compositions, methods, and kits for the treatment of degenerative joint disease such as osteoarthritis, that may be caused at least in part by the secretion of MMPs into the synovial fluid by synovial tissue.

In one aspect, the disclosure provides for a composition suitable for intra-articular injection comprising a first ion channel regulator and a second ion channel regulator different from the first ion channel regulator, and a pharmaceutically acceptable carrier. In some embodiments, the weight ratio of the first ion channel regulator to the second ion channel regulator is about 2:8 to about 8:2, about 3:7 to about 7:3, about 4:6 to about 6:4. For example, the weight ratio of the first ion channel regulator to the second ion channel regulator may be about 1:1. In another embodiment, the concentration of the combination of the first ion channel regulator and the second channel regulator is about 0.001 mg/ml to about 2 mg/ml.

Contemplated compositions may include a first ion channel regulator and a second ion channel regulator which are each independently selected from calcium channel regulators or sodium channel regulators. For example, the first ion channel regulator may be chosen from verapamil, amlodipine, diltiazem, bepridil, felodipine, gallopamil, isradipine, nicardipine, nimodipine, and nitrendipine, and the second ion channel regulator may be chosen from nifedipine, procainamide, quinidine, encainide, mexitil, disopyramide, and tetrodotoxin. In another embodiment, the first ion channel regulator may be chosen from nifedipine, amlodipine, diltiazem, bepridil, felodipine, gallopamil, isradipine, nicardipine, nimodipine, and nitrendipine, and the second ion channel regulator may chosen from verapamil, procainamide, quinidine, encainide, mexitil, disopyramide, and tetrodotoxin. A further exemplary embodiment includes a composition that includes a first ion channel regulator chosen from verapamil, amlodipine, nifedipine, bepridil, felodipine, gallopamil, isradipine, nicardipine, nimodipine, and nitrendipine, and a second ion channel regulator chosen from diltiazem, procainamide, quinidine, encainide, mexitil, disopyramide, and tetrodotoxin. For example, the first ion channel regulator and the second ion channel regulator can be selected from the group consisting of verapamil, diltiazem, and nifedipine. Certain contemplated compositions may further comprise a third ion channel regulator different from the first and second ion channel regulators.

Compositions provided herein may include a first and a second ion channel regulators in a synergistic combination for regulating matrix metalloproteinases in a joint of a patient. For example, upon administration of a disclosed composition to a patient suffering from a degenerative joint disease, the composition may be more effective than administering a composition that consists essentially of one ion channel regulator.

In certain embodiments, contemplated compositions may be substantially liquid at room temperature. For example, a composition may include a pharmaceutically acceptable carrier that comprises an aqueous solution and/or a polymer, for example polyethylene glycol. Contemplated compositions may include a pharmaceutically acceptable carrier that includes an aqueous solution (e.g. water) and/or polyethylene glycol and/or glycerin.

Also provided herein is a method for treating a degenerative joint disease, e.g. osteoarthritis, comprising administering a synergistically effective amount of a first ion channel regulator and a second ion channel regulator effective to treat the joint disease. In an embodiment, the first ion channel regulator and the second ion channel regulator are administered substantially simultaneously. In another embodiment, the first ion channel regulator and the second ion channel regulator are administered sequentially. The method may include administering a first and second ion channel regulator intra-articularly by injection.

Contemplated methods may further comprise administering an effective amount of another degenerative joint disease treatment agent, for example, another degenerative joint disease treatment agent is selected from the group consisting of viscosupplements, corticosteroids, non-steroidal anti-inflammatory agents, analgesics, glucosamines, chondroitin, and antibiotics.

Also provided herein is a method of protecting against collagen loss in a joint of a patient at risk of developing collagen loss, comprising administering a first ion channel regulator and a second ion channel regulator different from the first ion channel regulator. A method of reducing collagen loss in a joint of a patient in need thereof is also contemplated herein, wherein the method comprises administering intra-articularly a first ion channel regulator and a second ion channel regulator different from the first ion channel regulator, and for example, wherein the collagen loss of the patient after said administration is substantially less as compared to a patient not administered an ion channel regulator intra-articularly.

A kit for use in treating osteoarthritis is provided herein, wherein the kit comprises a container (e.g. a vial or a syringe) wherein a first ion channel regulator and a second ion channel regulator different than the first ion channel regulator are disposed in said container; and optionally, instructions for use.

### BRIEF DESCRIPTION OF THE DRAWINGS

FIG. 1 depicts the reduction of MMP activity in explanted synovial tissue of a 66 year old male patient incubated with IL-1β following contact with verapamil, diltiazem, and both verapamil and diltiazem.

FIG. 2 depicts the reduction of MMP activity in explanted synovial tissue of a 76 year old female patient incubated with IL-1β following contact with verapamil, diltiazem, and both verapamil and diltiazem.

FIG. 3 depicts the reduction of MMP activity in explanted synovial tissue of a 69 year old female patient incubated with IL-1β following contact with verapamil, diltiazem, and both verapamil and diltiazem.

FIG. 4 depicts the reduction of MMP activity in explanted synovial tissue of a 67 year old male patient incubated with IL-1β following contact with verapamil, diltiazem, and both verapamil and diltiazem.

FIG. 5 depicts the reduction of MMP activity in explanted synovial tissue of a 62 year old male patient incubated with IL-1β following contact with verapamil, diltiazem, and various concentrations of a combination of verapamil and diltiazem.

FIG. 6 depicts the reduction of MMP activity in explanted synovial tissue of a 83 year old female patient incubated with IL-1β following contact with verapamil, diltiazem, and various concentrations of a combination of verapamil and diltiazem.

FIG. 7 depicts the reduction of MMP activity in explanted synovial tissue of a 48 year old female patient incubated with IL-1β following contact with verapamil, diltiazem, and various concentrations of a combination of verapamil and diltiazem.

FIGS. 8-11 depict the reduction of MMP activity in explanted synovial tissue of a 73 year old female patient (FIG. 8), a 45 year old male patient (FIG. 9), a 71 year old female patient (FIG. 10) and a 78 year old female patient (FIG. 11) incubated with IL-1β following contact with verapamil, nifedipine, and both verapamil and nifedipine.

FIGS. 12-14 depicts the reduction of MMP activity in explanted synovial tissue of a 83 year old female patient (FIG. 12), a 59 year old female patient (FIG. 13) and a 67 year old female patient (FIG. 14) incubated with IL-1β following contact with verapamil, nifedipine, and various concentrations of a combination of verapamil and nifedipine.

FIGS. 15-18 and FIG. 18a depict the reduction of MMP activity in explanted synovial tissue of a 63 year old female patient (FIG. 15), a 78 year old female patient (FIG. 16), a 44 year old female patient (FIG. 17), a 61 year old male patient (FIG. 18) and a 57 year old male patient incubated with IL-1β following contact with nifedipine, diltiazem, and both diltiazem and nifedipine.

FIGS. 19-21 depicts the reduction of MMP activity in explanted synovial tissue of a 78 year old male patient (FIG. 19), a 72 year old male patient (FIG. 20) and a 77 year old female patient (FIG. 21) incubated with IL-1β following contact with verapamil, nifedipine, and various concentrations of a combination of verapamil and nifedipine.

FIGS. 22-25 depict the reduction of MMP activity in explanted synovial tissue of a 70 year old male patient (FIG. 22), a 64 year old female patient (FIG. 23), a 70 year old female patient (FIG. 24) and a 58 year old female patient (FIG. 25) incubated with IL-1β following contact with diltiazem, procainamide, and both diltiazem and procainamide.

FIG. 26 depicts the effect of verapamil and diltiazem on calcium current in HIG-82 cells. a) shows traces recorded at a membrane potential of −50 mV; b) traces recorded at 0 mV; each tracing was recorded using a different cell.

FIG. 27 indicates the inhibition of calcium current by verapamil and diltiazem.

FIG. 28 depicts the combination dose matrix showing combined effect of verapamil and diltiazem at six different concentrations, including 0; measured inhibition expressed as percent shown.

FIG. 29 depicts synergistic effect of verapamil and diltiazem; a) calculated excess inhibition over the predicted Bliss additivism model (predicted Bliss additive effect was subtracted from experimentally observed inhibition at each pair of concentrations); b) calculated excess inhibition over HSA (highest single agent).

FIG. 30 depicts protection from collagen loss in a menisectomy model of osteoarthritis in the rat.

### DETAILED DESCRIPTION

This disclosure is directed generally to compositions that include a first ion channel regulator and a second ion channel regulator different from the first ion channel regulator, and pharmaceutically acceptable carrier. In certain embodiments, the weight ratio of the first ion channel regulator to the second ion channel regulator may be selected so that the combination results in more than an additive physiological effect, e.g. for the treatment of inflammatory diseases such a degenerative joint disease, e.g. osteoarthritis.

As previously described, MMPs are believed to be primarily responsible for the destruction of joint cartilage that leads to joint pain associated with osteoarthritis. Specific levels of MMPs in joint synovial fluid that are believed to be regulated by interleukin-1 (IL-1), include MMP-1, also known as collagenase-1; MMP-2, also known as gelatinase A; MMP-3, also known as 15 stromelysin-1; MMP-8, also known as collagenase-2; and MMP-13, also known as collagenase-3. The levels of MMP activity produced by synovial tissue from patients having osteoarthritis is generally greater than the corresponding level obtained from patients who do not have arthritis. Ion-channel regulators, e.g. calcium-channel regulators, may be capable of interfering with the effect of IL-1 on synovial cells, and may be useful in treating osteoarthritis. See, for example, co-pending U.S. patent applications Ser. Nos. 11/138,738 and 11/138,744, hereby incorporated by reference.

Before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

The term “therapeutic effect” is art-recognized and refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and/or conditions in an animal or human. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease condition being treated, the weight and age of the subject, the severity of the disease condition, the manner of administration and the like, which can readily be determined by one of ordinary skill in the art. For example, certain compositions of the present invention may be administered in a sufficient amount to produce a at a reasonable benefit/risk ratio applicable to such treatment.

A “patient,” “subject” or “host” to be treated by the subject method may mean either a human or non-human animal.

The term “treating” is art-recognized and refers to curing as well as ameliorating at least one symptom of any condition or disease.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier or excipient must be “acceptable” in the sense of being compatible with the subject composition and its components and not injurious to the patient. Some examples of materials which may serve as pharmaceutically acceptable carriers include: (1) sugars, such as lactose, glucose and sucrose, and derivatives and/or polymers or co-polymers thereof; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) other excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. Other carriers useful in compositions disclosed herein are described below.

The term “ion-channel regulator” can refer to ion-channel blockers and/or ion-channel activators, and refers to any agent that can alter the entry of certain ions into or out of cells and cellular organelles, depending on whether the intracellular or extracellular concentration of the particular ion is greater and the electrical potential difference that exists between the inside and the outside of the cell. Exemplary ion-channel regulators include calcium-channel regulators, sodium-channel regulators, a potassium-channel regulators, chloride-channel regulators, cation-ion channel regulators, anion-ion channel regulators, non-selective ion channel regulators, mixed channel regulators, and connexon-channel regulators (i.e., chemical agents that regulate the movement of ions and molecules through connexons in synovial cells, which consist of the protein known as connexin 43). Calcium-, sodium-, potassium-, chloride-, cation- and anion-channel regulators respectively substantially regulate the movement of calcium ions, sodium ions, potassium ions, chloride ions, anions and cations through ion channels in the membranes of cells. Non-selective ion channels are ion channels that allow any combination of anions and cations to pass through the membranes of cells, and non-selective ion-channel regulators regulate the movement of those ions. Connexon-channel regulators regulate the movement of ions through connexons.

The term “synergistic” is art recognized and refers to two or more components working together so that the total effect is greater than the sum of the components.

In some embodiments, compositions contemplated herein comprise a combination of at least two different ion-channel regulators. For example, a first ion-channel regulator may regulate certain ions passing through ion channels in the synovial cells. A second ion-channel regulator may regulate other ions passing through the ion channels that are not regulated by the first ion-channel regulator, and/or may regulate the same ions but in a different physiological way. When administered to synovial tissue, for example, the combined ion-channel regulators may provide a e.g. beneficial effect on the underlying cellular processes, for example, to those processes that lead directly or indirectly to e.g., pain and tissue destruction associated with osteoarthritis, and/or may, e.g. inhibit production of MMPs. In certain embodiments, quite unexpectedly been found that e.g. effects on cellular processes e.g production of MMPs, provided by a combination of ion-channel regulators is greater than either ion-channel regulator provides separately. In certain embodiments, certain effects of a combination of a first ion-channel regulator and a second ion-channel regulator may also be greater than the beneficial effect that would be expected if the individual beneficial effects of each ion channel regulator were added together, e.g. a synergistic effect.

In certain embodiments, a composition is provided comprising a first ion channel regulator and a second ion channel regulator, wherein the first and second ion channel regulators are selected from the group consisting of sodium channel regulators and calcium channel regulators. Representative examples of calcium-channel regulators include verapamil, nifedipine, diltiazem, amlodipine, bepridil, felodipine, gallopamil, isradipine, nicardipine, nimodipine, nitrendipine, and mixtures thereof. Representative examples of sodium-channel regulators include procainamide, quinidine, encainide, mexitil, disopyramide, tetrodotoxin, and mixtures thereof.

For example, compositions provided herein may comprise combinations of two or more calcium or sodium ion-channel regulators; e.g. two or more calcium-channel regulators, or two or more sodium-channel regulators; or may comprise combinations of e.g., one or more calcium-channel regulators in combination with one or more sodium-channel regulators. Without intending to be bound by theory, combining at least one calcium-channel blocker and at least one sodium-channel blocker in the composition may provide e.g. a synergistic beneficial effect by acting to block both of the types of the ion channels known to be involved in MMP production. When e.g. two calcium-channel blockers or two sodium-channel blockers are combined, synergistic beneficial results may be provided because each of the ion-channel regulators bind to a different place on the ion channel and thereby each block the passage of ions that are not blocked by the other similar ion-channel regulator. For example, contemplated compositions include a composition comprising verapamil and diltiazem, a composition comprising verapamil and nifedipine, and composition comprising nifedipine and diltiazem. In another embodiment, a composition comprising a combination of a calcium-channel regulator and a sodium-channel regulator is provided, e.g. a composition comprising diltiazem and procainamide.

Compositions provided herein may include e.g. two calcium channel regulators and a non-calcium channel regulator. For example, a composition is provided that includes procainamide, verapamil and nifedipine. Such a composition may e.g. increase a synergistic effect over that produced by the combination of verapamil and nifedipine alone. Other exemplary compositions may include those that include a combination of verapamil, diltiazem and procainamid.

In an embodiment, a contemplated composition comprises a combination of ion-channel regulators having a total concentration of about 0.001 mg/mL to about 2 mg/mL, about 0.01 mg/mL to about 2 mg/mL, about 0.05 mg/mL to 1.5 mg/mL, or about 1.0 mg/mL. For example, administration of a composition that includes a ion-channel concentration of about 1 mg/mL (e.g. a composition that includes both about 0.5 mg verapamil and about 0.5 mg nifedipene per mL of a pharmaceutically acceptable excipient or carrier) may provide an effective treatment when the composition is administered to synovial cells.

In some embodiments, a composition contemplated herein may include a first ion channel regulator and a second ion channel regulator, wherein the weight ratio of the first ion channel regulator to the second ion channel regulator is about 1:9 to about 9:1, or about 2:8 to about 8:2, or about 3:7 to about 7:3, or about 4:6 to about 6:4. For example, a composition suitable for intra-articular injection is provided that includes a first ion channel regulator and a second ion channel regulator different from the first ion channel regulator, wherein the weight ratio of the first ion channel regulator to the second ion channel regulator is about 2:8 to about 8:2, and a pharmaceutically acceptable excipient.

In some embodiments, the weight ratio of the first ion channel regulator to the second ion channel regulators is about 1:1. For example, a contemplated composition may include 50% of the total ion channel regulators (e.g. verapimil) by weight and 50% of the total ion channel regulators (e.g. diltiazem by weight. In certain other exemplary embodiments, a contemplated composition may include a 50%/50% by weight of verapamil and nifedipine, each having a concentration of about 0.5 mg/ml in the composition.

In some embodiments, it is contemplated that the composition comprising the combination of ion-channel regulators in accordance with the present invention will be significantly more effective in reducing pain and improving function in a diseased joint than would either regulator used alone, or that would be expected from adding together the demonstrated effects of each regulator.

The compositions of the present invention further comprise a pharmaceutically acceptable carrier for the ion-channel regulators that is suitable for intra-articular injection, i.e. injection directly into the closed joint cavity. Intra-articular injection may allow biologically sufficient concentrations of the ion-channel regulators to be applied to the affected synovial tissue without the risk of producing undesirable side-effects that can occur as the result of higher concentrations of ion-channel regulators.

Suitable carriers that form part of the contemplated compositions may include substantially liquid carriers, e.g. water-based (aqueous) carriers such as distilled water, saline solutions and the like.

Ion-channel regulators useful in the compositions of the invention may either be soluble or insoluble in pharmaceutically acceptable carrier component, e.g. in a aqueous carrier component. For example, diltiazem and procainamide are mostly soluble in aqueous carriers, nifedipine is substantially insoluble in an aqueous carrier, and verapamil exists in both aqueous soluble and insoluble forms. In certain embodiments, compositions are provided that comprise a combination of soluble ion-channel regulators in accordance with the invention, e.g. verapamil and diltiazem, or verapamil, diltiazem and procainamide, and an pharmaceutically acceptable carrier comprises e.g. water. Such a composition may be substantially stable (e.g. for up to or more than 6 months).

In other embodiments, a pharmaceutically acceptable carrier contemplated herein includes a polymer and an aqueous solution (e.g. water). For example, an insoluble ion-channel regulator may form microcrystals which may have varying sizes characteristic of a particular ion-channel regulator, i.e. a first insoluble ion-channel regulator may form microcrystals of one size and a second insoluble ion-channel regulator may form microcrystals of a different size. Therefore, in a composition comprising a combination of a soluble ion-channel regulator such as verapamil and an insoluble ion-channel regulator such as nifedipine, the soluble verapamil may remain in solution, but the insoluble nifedipine may exist in the form of microcrystals in suspension. Such suspensions may be unstable because the microcrystals tend to aggregate and precipitate out. Thus, the carrier used for compositions of the invention comprising combinations of soluble and insoluble ion-channel regulators is in some embodiments, formulated to assure that the composition is stable and that any microcrystals if present, are of a sufficiently small size to allow the composition to be injected through needles commonly used for intra-articular injection. For example, in an embodiment, the composition of the invention comprises a carrier comprising a combination of polyethylene glycol and water and optionally glycerin or the like, which may stabilize the ion-channel regulators in, e.g. a suspension. In some embodiments, the pharmaceutically acceptable carrier components are chosen to control the size and/or the formation of microcrystals of any insoluble ion-channel regulators in the contemplated composition, if present. For example, control of microparticle size may maintain the suitability of the composition for intra-articular injection. The presence of such microparticles may provide an added benefit of remaining in the joint longer than soluble ion-channel regulators once administered.

Particle size and/or presence may be controlled using various techniques. For example, a combination of two insoluble ion channel particles may be present in an an aqueous carrier (for example, saline, in a syringe). Since both ion-channel regulators are insoluble, they may be present as a residue of small particles at the bottom of the syringe, and as such are not immediately suitable for injection. However, the syringe could be placed in a device, for example an ultrasonic vibrator, that may e.g. instantly generate microparticles. The microparticles could then easily be injected and may be present as microparticles in the joint and e.g. dissolve substantially slowly. Other methods and techniques for producing the controlled release of the ion channel regulators are known to those skilled in the art. As non-limiting examples, such methods are described in Grayson A C R, Choi I S, Tyler B M, Wang P P, Brem H, Cima M I, Langer R, Multi-pulse Drug Delivery From a Resorbable Polymeric Microchip Device, Nature Materials 2, 767-772, 2003, and Lassalle V, Ferreira M L, PLA Nano- and Microparticles for Drug Delivery: Overview of the Methods of Preparation, Macromolecular Bioscience 7:767-783, 2007; both of which are incorporated by reference herein.

Compositions disclosed herein may also contain other materials such as fillers, stabilizers, coatings, coloring agents, preservatives, fragrances, and other suitable additives. As one example, the composition may include a substance that provides for the slow release of the ion-channel blockers in the composition such as, for example, polylactic acid, poly glycolic acid, collagen, nanoparticles, etc.

Kits for use in treating arthritis, e.g. osteoarthritis, are also contemplated. An exemplary kit may include a first ion channel regulator and a second ion channel regulator disposed in one container, or each disposed in separate containers. A needle may also be provided for ease of use. Optionally instructions for use are included within the kit. In some embodiments, the container is a syringe or vial.

In one embodiment, the present invention provides a method of treating a degenerative joint disease which comprises administering, e.g. by injecting into a diseased joint, a first ion channel regulator and a second ion channel regulator. The first ion channel regulator and the second ion channel regulator may be administered sequentially, i.e. administered as a first composition that includes the first ion channel regulator and then as a second composition than includes the second ion channel regulator, or a composition comprising a combination of at least two ion-channel regulators. The two ion channel regulators may be administered so that the resultant concentration is sufficient to provide an effective amount of the ion-channel regulators in the synovial fluid. For example, the resultant concentration may be a synergistic amount of the first and second ion channel regulator. It is understood that the effective amount will typically vary depending on the level of disease in the joint and the joint being treated. An effective amount may sometimes be expressed as the weight in milligrams of the ion-channel regulator combination that is administered per kilogram of patient body weight (also sometimes referred to as a “dose”). For example, it is known that joints contain differing amounts of synovial fluid, depending on the extent of osteoarthritis in the joint and the size of the joint. For example, see Waddell, D. D. and Marino, A. A., Chronic knee effusions in patients with advanced osteoarthritis: implications for functional outcome of viscosupplementation; J. Knee Surg; 2007, which is incorporated by reference herein. For example, when the composition(s) comprising the ion channel regulator(s) combination is/are injected directly into a joint, the concentration of ion-channel regulators in the composition will be diluted by the amount of the synovial fluid in the joint. Accordingly, in an embodiment, the concentration of ion-channel regulators in the composition(s) as injected may have to be sufficiently high to elevate the total concentration of ion-channel regulators in the synovial fluid to e.g., about 0.01 mg/ml to about 2 mg/ml after injection of the composition into the joint. Exemplary amounts of contemplated compositions administered may varying by the size and type of the affected joint.

In some embodiments, methods for degenerative joint disease contemplated here include methods for treating arthritis, e.g. osteoarthritis, rheumatoid arthritis and psoriatic arthritis. CAL-002

Other joints in the human body typically have an amount of synovial fluid that is less than the volume of synovial fluid present in the knee joint. Using the principles described above, one skilled in the art may easily calculate the desired dose of the ion-channel regulator combination of the invention for the treatment of various levels of osteoarthritis in these other joints, as well calculate the total concentration of ion-channel regulators in the composition of the invention that is used for the intra-articular injection of the joint.

Also contemplated herein is a method of protecting and or reducing collagen loss in a joint of a patient at risk of developing collagen loss comprising administering, e.g. intra-articularly (e.g. by injection) a first ion channel regulator and second ion channel regulator. In some embodiments, collagen loss is substantially less in a patient that has received administration of two or more ion channel regulators as compared to a patient who has not been administered an ion channel regulator intra-articularly, or has been administered only one ion channel regulator intra-articularly.

Methods contemplated herein may further include administration of one or more other agents suitable for the treatment of degenerative joint disease, either in separate compositions or as a component of the same composition. The treatment method of the present invention may readily be customized to the individual patient's needs, and may be used instead of or in conjunction with other treatment modalities including but not limited to physical therapy, treatments that provide localized pain relief (heat, massage, application of liniments, etc.), and with other medications that help reduce disability, relieve pain, and improve the patient's quality of life. Accordingly, methods of treatments contemplated to be within the present invention may include an intraarticular injection of a composition of the present invention followed by another intraarticular injection of another osteoarthritis treatment agent, e.g. a viscosupplement, steroid or other injectable osteoarthritis treatment agent; an intraarticular injection of a composition of the present invention followed by oral or intravenous administration of another osteoarthritis treatment agent such as a non-steroidal antiinflammatory drug; an intraarticular injection of a single composition comprising at least two ion-channel regulators in accordance with the present invention and at least one viscosupplement, steroid or other injectable osteoarthritis treatment agent; and so forth.

As used herein and in the art, the term “viscosupplement” refers to any substance that is used to restore and/or increase the cushioning and lubrication of arthritic synovial fluid by intraarticular injection. Preferred viscosupplements include hylan, hyaluronic acid and other hyaluronan (sodium hyaluronate) compounds, which are natural complex sugars of the glycosaminoglycan family. Hyaluronan, in particular, is a long-chain polymer containing repeating disaccharide units of Na-glucoronate-N-acetylglucosamine. By way of example, commercially available hyaluronan visco supplements include Synvisc®, Hyalgan®, Supartz®, and Orthovisc®.

Examples of other osteoarthritis treatment agents include, without limitation, nonsteroidal anti-inflammatory drugs (NSAIDS) such as ibuprofen, naproxen, and COX-2 inhibitors; analgesics such as aspirin and acetaminophen; glycans, including glucosamines, e.g. glucosamine sulfate and glucosamine hydrochloride; and proteoglycans, such as chondroitin compounds, as well as various other known narcotics, steroids, antibiotics, immunomodulators, penicillamine, and the like.

A contemplated composition provided herein, may, in some embodiments, increase joint function and/or decrease pain in a patient. Joint pain may be assessed indirectly or directly. Indirect measures of joint pain and/or joint function include static and dynamic weight-bearing, foot posture, and/or gait analysis including e.g. extremity (e.g. hand or paw) elevation time during walking, spontaneous mobility, and/or heat sensitivity.

For example, measurements of weight bearing in animal models include analysis of weight distribution on e.g. two hind paws, or a leg/foot) as a measure of the force exerted by each limb on a transducer plate in the floor over a given time period, with weight borne by each hind limb expressed e.g., as percent of body weight, percent of weight borne by both hind limbs, or the ratio or difference between each hind limb. A significant shift of weight from the arthritic site to the contralateral limb can be taken as a pain measure. Weight bearing and gait analysis are used for example in clinical settings as well.

Posture, range of motion gait analysis can be quantified in arthritis models using rating scales, e.g. by analyzing static (standing) and dynamic (walking) behaviors to calculate a pain score in animals and/or patients with joint arthritis.

Behavioral tests may also be used to ascertain pain, stiffness, and/or function by directly assessing mechanical sensitivity of a joint, e.g. a knee joint, by measuring hind limb withdrawal reflex threshold of knee compression force, struggle threshold angle of knee extension and/or vocalizations evoked by stimulation of the knee. Clinically, patients can also be assessed for pain using patient self report questionnaires such as the Visual Analog Scale (VAS), McGill Pain Questionnaire (MPQ), Western Ontario and McMaster Universities Osteoarthritis Index (WOMAC), Health Assessment Questionnaire (HAQ), Medical Outcomes Study 36-item Short Form Health Survey (SF-36), and Disease Activity Score (DAS-28). For example, the WOMAC is one of the most commonly used measures of pain and/or physical disability (e.g. joint function) in patients with osteoarthritis of the hip and/or knee, with demonstrated reliability and validity in a range of patient groups. The WOMAC evaluates three dimensions (pain, stiffness and physical function) using a numeric rating scale or VAS. Typically, in addition to a score of each subscale, an index score or global score is calculated.

The following Examples using the above-described test procedures demonstrate the synergistic results achieved by administering a composition of the present invention to human synovial tissue:

### EXAMPLE 1

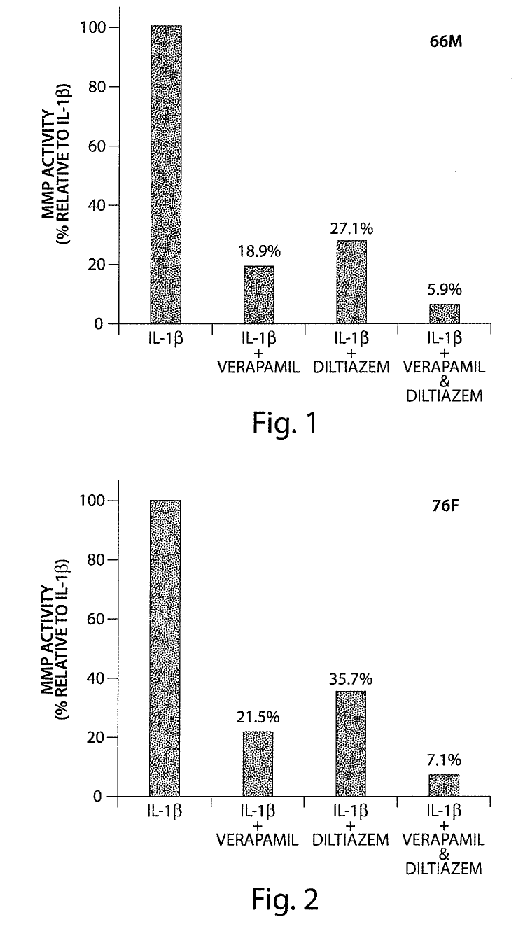

Multiple samples of synovial tissue, each approximately 20 mg, were obtained from a 66-year-old male with osteoarthritis (grade IV, Kellgren-Lawrence scale). The amount of MMPs produced in culture by the tissue when it was incubated with the pro inflammatory agent interleukein-If (IL-1β) was defined to be 100% (depicted by the first bar in FIG. 1, labeled “IL-1β”). When the calcium-channel regulator verapamil was added at a concentration of 0.01 mg/mL, the MMPs produced in response to IL-1β were partially blocked, with the result that the activity of the MMPs was reduced to 38% of that measured in tissues not exposed to the regulator. Increasing the concentration of verapamil between 0.01 mg/mL and 0.05 mg/mL resulted in a progressive increase in the inhibition o MMP activity. At 0.05 mg/mL the MMPs were reduced to 18.9% (second bar in FIG. 1). Further increasing the concentration up to 0.1 mg/mL had no further effect on blocking MMP activity below the level obtained using a concentration of 0.05 mg/mL of the regulator. At a concentration above 0.1 mg/mL, the death of cells in the synovial tissue was observed using the trypan blue exclusion test: which consists of adding an appropriate amount of trypan blue dye to the environment of the cells; cells are able to exclude the dye if they are healthy, but if they are injured or dead, the dye enters the cell and stains it blue thereby detecting harmful and lethal effects of certain high levels of ion-channel regulators on cells.

When the calcium-channel regulator diltiazem was applied at a concentration of 0.01 mg/mL, the MMPs produced in response to IL-1β were partially blocked with the result that the activity of the MMPs was 97% of that measured in tissues not exposed to the regulator. Increasing the concentration of diltiazem between 0.01 mg/mL and 0.05 mg/mL resulted in a progressive increase in the inhibition of MMP activity. At 0.05 mg/mL the MMPs were reduced to 27.1% (third bar in FIG. 1). Further increasing the concentration up 15 to 0.1 mg/mL had no further effect on blocking MMP activity below the level obtained using a concentration of 0.05 mg/mL of the regulator. At a concentration above 0.1 mg/mL, the death of cells in the synovial tissue was observed using the trypan blue exclusion test.

When synovial tissue from the same patient was exposed to a combination of verapamil and diltiazem, each at a concentration of 0.05 mg/mL, the MMPs produced under standard incubation conditions were reduced to 5.9% (fourth bar in FIG. 1). It can be seen that each of the two ion-channel regulators was partially effective in reducing the amount of MMPs produced, and the combination of the two regulators was more effective in reducing MMPs than either regulator alone. While not intending to be bound by theory, it is believed that diltiazem regulated ions passing through some calcium channels that were not regulated by verapamil, thereby producing a synergistic effect on the inhibition of MMP activity, which was reduced from 18.9% to 5.9%.

### EXAMPLE 2

Multiple samples of synovial tissue, each approximately 20 mg, were obtained from a 76-year-old female with osteoarthritis (grade IV, Kellgren-Lawrence scale). The amount of MMPs produced in culture by the tissue when it was incubated with the pro-inflammatory agent interleukein-1β (IL-1β) was defined to be 100% (depicted by the first bar in FIG. 2, labeled “IL-1β”). When the calcium-channel regulator verapamil was added at a concentration of 0.01 mg/mL, the MMPs produced in response to IL-1β were partially blocked, with the result that the activity of the MMPs was reduced to 37.2% of that measured in tissues not exposed to the regulator. Increasing the concentration of verapamil between 0.01 mg/mL and 0.05 mg/mL resulted in a progressive increase in the inhibition of MMP activity. At 0.05 mg/mL the MMPs were reduced to 21.5% (second bar in FIG. 2). Further increasing the concentration up to 0.1 mg/mL had no further effect on blocking MMP activity below the level obtained using a concentration of 0.05 mg/mL of the regulator. At a concentration above 0.1 mg/mL the death of cells in the synovial tissue was observed, using the trypan blue exclusion test.

When the calcium-channel regulator diltiazem was applied at a concentration of 0.01 mg/mL, the MMPs produced in response to IL-1β were partially blocked with the result that the activity of the MMPs was 99% of that measured in tissues not exposed to the regulator. Increasing the concentration of diltiazem between 0.01 mg/mL and 0.05 mg/mL resulted in a progressive increase in the inhibition of MMP activity. At 0.05 mg/mL the MMPs were reduced to 35.7% (third bar in FIG. 2). Further increasing the concentration up to 0.1 mg/mL had no further effect on blocking MMP activity below the level obtained using a concentration of 0.05 mg/mL of the regulator. At a concentration above 0.1 mg/mL the death of cells in the synovial tissue was observed, using the trypan blue exclusion test.

When synovial tissue from the same patient was exposed to a composition comprising a combination of verapamil and diltiazem, each at a concentration of 0.05 mg/mL, the MMPs produced under standard incubation conditions were reduced to 7.1% (fourth bar in FIG. 2). As in Example 1 above, each of the two ion-channel regulators was partially effective in reducing the amount of MMPs produced (verapamil to 21.5%; 19 diltiazem to 35.7%), whereas the combination of the two regulators was synergistically more effective in reducing MMPs (to 7.1%).

### EXAMPLE 3

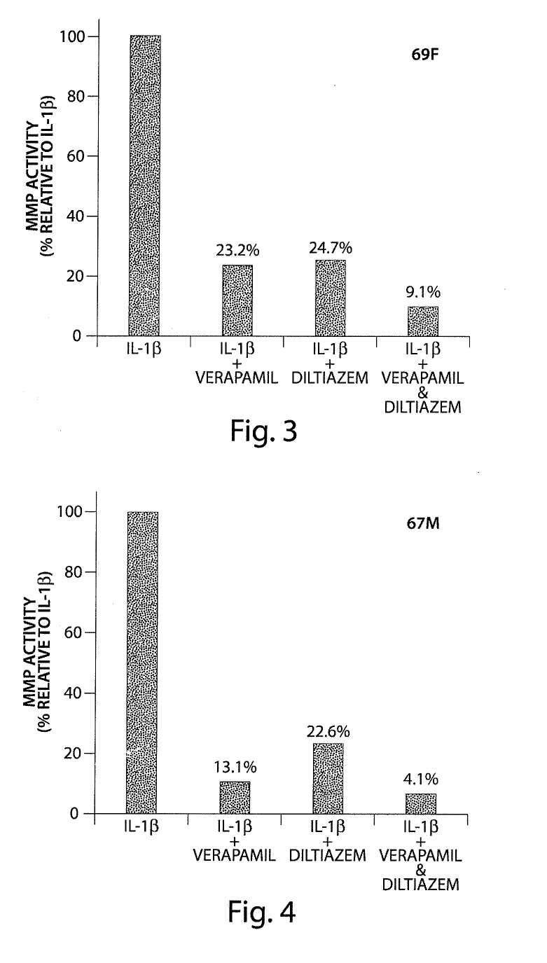

Multiple samples of synovial tissue, each approximately 20 mg, were obtained from a 69-year-old female with osteoarthritis (grade IV, Kellgren-Lawrence scale). The amount of MMPs produced in culture by the tissue when it was incubated with the pro-inflammatory agent interleukin-If (IL-1β) was defined to be 100% (depicted by the first bar in FIG. 3, labeled “IL-1β”). When the calcium-channel regulator verapamil was added at a concentration of 0.01 mg/mL, the MMPs produced in response to IL-1β were partially blocked, with the result that the activity of the MMPs was reduced to 37% of that measured in tissues not exposed to the regulator. Increasing the concentration of verapamil between 0.01 mg/mL and 0.05 mg/mL resulted in a progressive increase in the inhibition of MMP activity. At 0.05 mg/mL the MMPs were reduced to 23.2% (second bar in FIG. 3). Further increasing the concentration up to 0.1 mg/mL had no further effect on blocking MMP activity below the level obtained using a concentration of 0.05 mg/mL oft he regulator. At a concentration above 0.1 mg/mL the death of cells in the synovial tissue was observed, using the trypan blue exclusion test.

When the calcium-channel regulator diltiazem was applied at a concentration of 0.01 mg/mL, the MMPs produced in response to IL-1β were partially blocked with the result 20 that the activity of the MMPs was 97% of that measured in tissues not exposed to the regulator. Increasing the concentration of diltiazem between 0.01 mg/mL and 0.05 mg/mL resulted in a progressive increase in the inhibition of MMP activity. At 0.05 mg/mL the MMPs were reduced to 24.7% (third bar in FIG. 3). Further increasing the concentration up to 0.1 mg/mL had no further effect on blocking MMP activity below the level obtained using 25 a concentration of 0.05 mg/mL of the regulator. At a concentration above 0.1 mg/mL the death of cells in the synovial tissue was observed, using the trypan blue exclusion test.

When synovial tissue from the same patient was exposed to a composition comprising a combination of verapamil and diltiazem, each at a concentration of 0.05 mg/mL, the MMPs produced under standard incubation conditions were reduced to 9.1% (fourth bar in FIG. 3). As in the previous examples above, each of the two ionchannel regulators was partially effective in reducing the amount of MMPs produced 20 (verapamil to 23.2%; diltiazem to 24.7%), whereas the combination of the two regulators was synergistically more effective in reducing MMPs (to 9.1%).

### EXAMPLE 4

Multiple samples of synovial tissue, each approximately 20 mg, were obtained from a 67-year-old male with osteoarthritis (grade IV, Kellgren-Lawrence scale). The amount of MMPs produced in culture by the tissue when it was incubated with the pro-inflammatory agent interleukin-1β (IL-1β) was defined to be 100% (depicted by the first bar in FIG. 4, labeled “IL-1β”). When the calcium-channel regulator verapamil was added at a concentration of 0.01 mg/mL, the MMPs produced in response to IL-1β were partially blocked, with the result that the activity of the MMPs was reduced to 35% of that measured in tissues not exposed to the regulator. Increasing the concentration of verapamil between 0.01 mg/mL and 0.05 mg/mL resulted in a progressive increase in the inhibition of MMP activity. At 0.05 mg/mL the MMPs were reduced to 13.6% (second bar in FIG. 4). Further increasing the concentration up to 0.1 mg/mL had no further effect on blocking MMP activity below the level obtained using a concentration of 0.05 mg/mL of the regulator. At a concentration above 0.1 mg/mL the death of cells in the synovial tissue was observed, using the trypan blue exclusion test.

When the calcium-channel regulator diltiazem was applied at a concentration of 0.01 mg/mL, the MMPs produced in response to IL-1β were partially blocked with the result 20 that the activity of the MMPs was 98.1% of that measured in tissues not exposed to the regulator. Increasing the concentration of diltiazem between 0.01 mg/mL and 0.05 mg/mL resulted in a progressive increase in the inhibition of MMP activity. At 0.05 mg/mL the MMPs were reduced to 22.6% (third bar in FIG. 4). Further increasing the concentration up to 0.1 mg/mL had no further effect on blocking MMP activity below the level obtained using a concentration of 0.05 mg/mL of the regulator. At a concentration above 0.1 mg/mL the death of cells in the synovial tissue was observed, using the trypan blue exclusion test.

When synovial tissue from the same patient was exposed to a composition comprising a combination of verapamil and diltiazem, each at a concentration of 0.05 mg/mL, the MMPs produced under standard incubation conditions were reduced to 4.1% (fourth bar in FIG. 4). As in the previous Examples above, each of the two ionchannel regulators was partially effective in reducing the amount of MMPs produced (verapamil to 13.1%; diltiazem to 22.6%), whereas the combination of the two regulators was synergistically more effective in reducing MMPs (to 4.1%).

### EXAMPLE 5

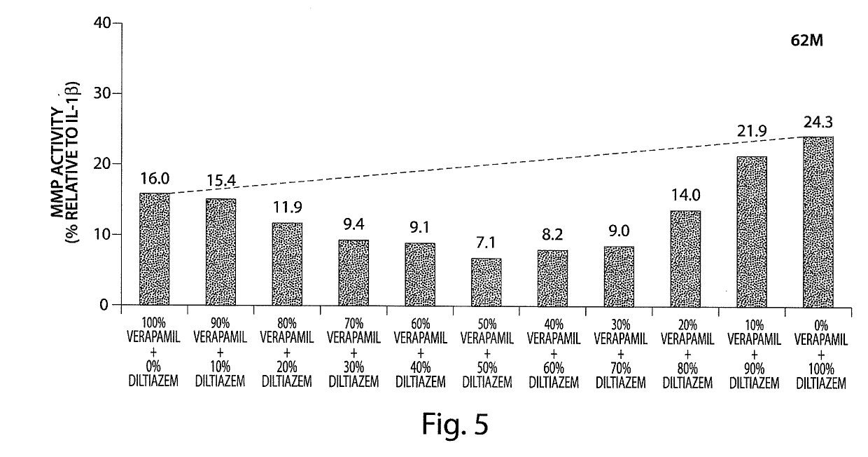

Multiple samples of synovial tissue, each approximately 20 mg, were obtained from a 62-year-old male with osteoarthritis (grade IV, Kellgren-Lawrence scale). The amount of MMPs produced in culture by the tissue when it was incubated with a pro-inflammatory agent interleukin-If (IL-1β) was defined to be 100%. When a composition comprising 0.1 mg/mL of the calcium-channel regulator verapamil was added, the MMPs produced in response to IL-1β were partially blocked, with the result that the activity of the MMPs was 16% of that measured in tissues not exposed to the regulator (first bar in FIG. 5). When 10% of the verapamil in the composition was replaced with diltiazem on an equal weight basis, the inhibition of MMPs decreased to 15.4% (second bar in FIG. 5). Further decreasing the amount of verapamil and increasing the amount of diltiazem in the composition such that the total concentration remained at 0.1 mg/mL resulted in a progressive increase in the inhibition of MMPs. The maximum inhibition (to 7.1%) occurred when the relative amounts of the regulators present in the composition was 50%-50% (sixth bar in FIG. 5). Further increasing the relative concentration of diltiazem resulted in a decrease in the inhibition of MMPs, and when the amount of verapamil was reduced to 0, a composition comprising diltiazem alone produced an MMP effect of only 24.8% (bars 7-11 in FIG. 5). The dotted line in FIG. 5 shows the expected results if the effect of verapamil and diltiazem were merely additive. The results shown in FIG. 5 clearly demonstrate that the effects on MMP production of combining the ion-channel regulators in the composition in accordance with the invention are synergistic rather than additive, and that the maximum synergistic effect occurred when a 50%-50% combination of the regulators was employed.

### EXAMPLE 6

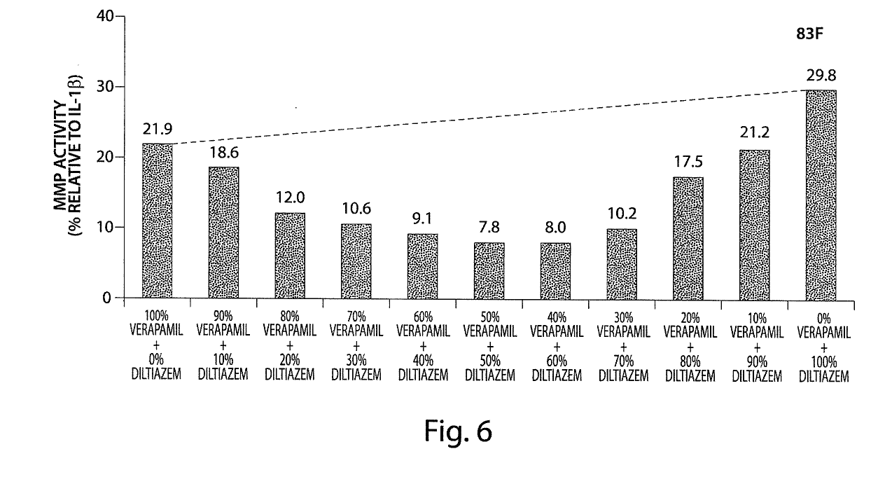

Multiple samples of synovial tissue, each approximately 20 mg, were obtained from an 83-year-old female with osteoarthritis (grade IV, Kellgren-Lawrence scale). The amount of MMPs produced in culture by the tissue when it was incubated with a pro-inflammatory agent interleukin-1β (IL-1β) was defined to be 100%. When a composition comprising 0.1 mg/mL of the calcium-channel regulator verapamil was added, the MMPs produced in response to IL-1β were partially blocked, with the result that the activity of the MMPs was 21.9% of that measured in tissues not exposed to the regulator. When 10% of the verapamil in the composition was replaced with diltiazem on an equal weight basis, the inhibition of MMPs decreased to 18.6% (second bar in FIG. 6). Further decreasing the amount of verapamil and increasing the amount of diltiazem in the composition such that the overall concentration remained at 0.1 mg/mL resulted in a progressive increase in the inhibition of MMPs. The maximum inhibition (to 7.8%) occurred when the relative amounts of the regulators present was 50%-50% (sixth bar in FIG. 6). Further increasing the relative concentration of diltiazem resulted in a decrease in the inhibition of MMPs, and when the amount of verapamil was reduced to 0, a composition comprising diltiazem alone produced an MMP effect of only 29.8% (bars 7-11 in FIG. 6). The dotted line in FIG. 6 shows the expected results if the effect of verapamil and diltiazem were merely additive. The results shown in FIG. 6 confirm that the effects on MMP production of combining the ion-channel regulators in accordance with the invention are synergistic rather than additive, and that the maximum synergistic effect occurred when a 50%-50% combination of the regulators was employed.

### EXAMPLE 7

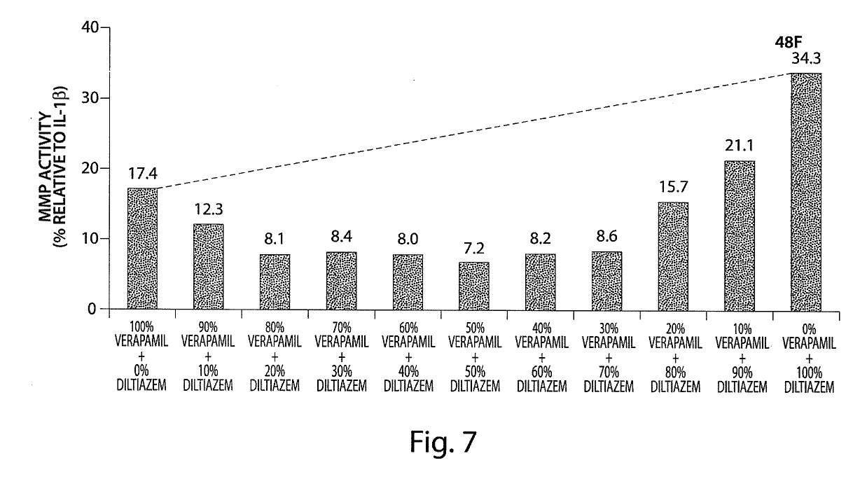

Multiple samples of synovial tissue, each approximately 20 mg, were obtained from a 48-year-old female with osteoarthritis (grade IV, Kellgren-Lawrence scale). The amount of MMPs produced in culture by the tissue when it was incubated with a pro-inflammatory agent interleukin-1β (IL-1β) was defined to be 100%. When a composition comprising 0.1 mg/mL of the calcium-channel regulator verapamil was added, the MMPs produced in response to IL-1β were partially blocked, with the result that the activity of the MMPs was 17.4% of that measured in tissues not exposed to the regulator. When 10% of the verapamil in the composition was replaced with diltiazem on an equal weight basis, the inhibition of MMPs decreased to 12.3% (second bar in FIG. 7). Further decreasing the amount of verapamil and increasing the amount of diltiazem in the composition such that the overall concentration remained at 0.1 mg/mL resulted in a progressive increase in the inhibition of MMPs. The maximum inhibition (to 7.2%) occurred when the relative amounts of the regulators present was 50%-50% (sixth bar in FIG. 7). Further increasing the relative concentration of diltiazem resulted in a decrease in the inhibition of MMPs, and when the amount of verapamil was reduced to 0, a composition comprising diltiazem alone produced an MMP effect of only 34.3% (bars 7-11 in FIG. 7). The dotted line in FIG. 7 shows the expected results if the effect of verapamil and diltiazem was merely additive. The results shown in FIG. 7 again confirm that the effects on MMP production of combining the ion channel regulators were synergistic rather than additive, and that the maximum synergistic effect occurred when a 50%-50% combination of the regulators was employed.

### EXAMPLES 8-21

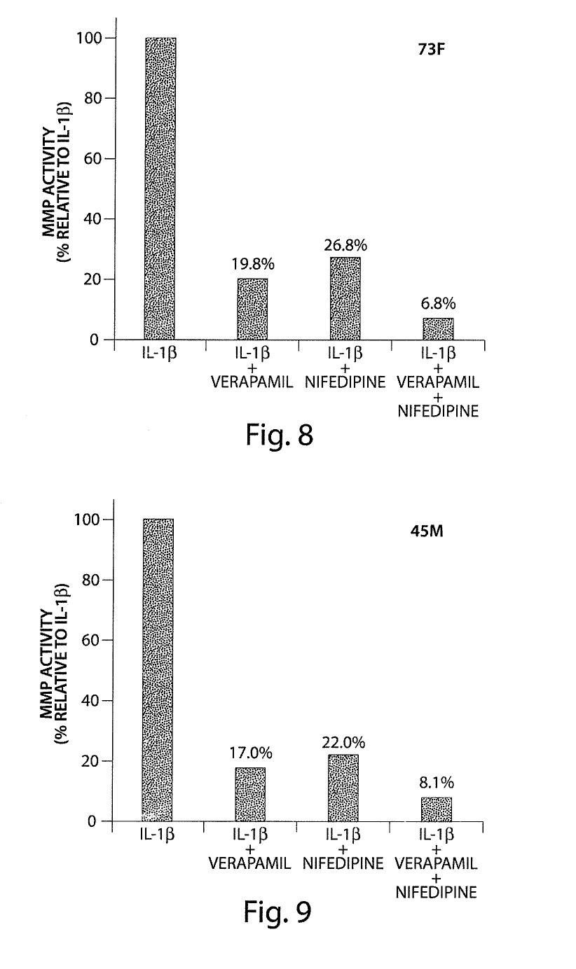

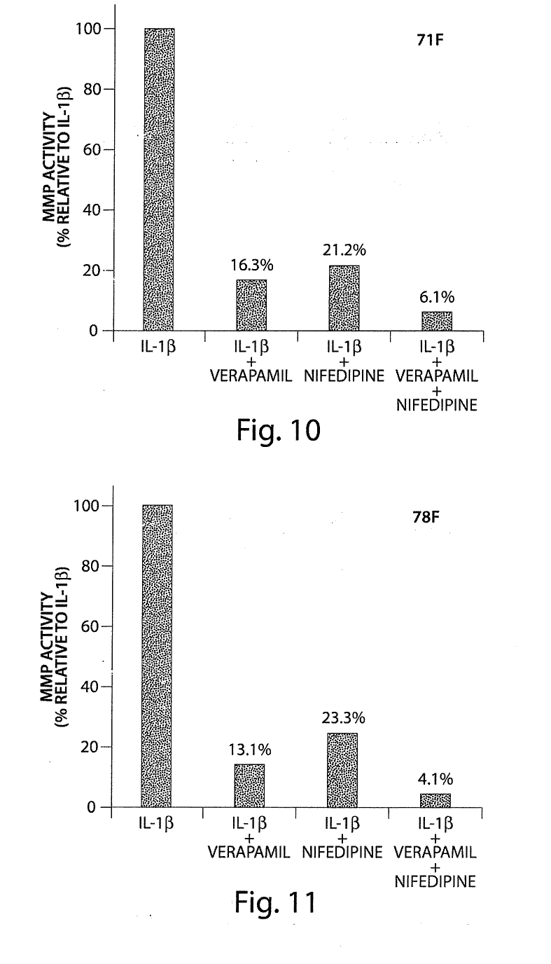

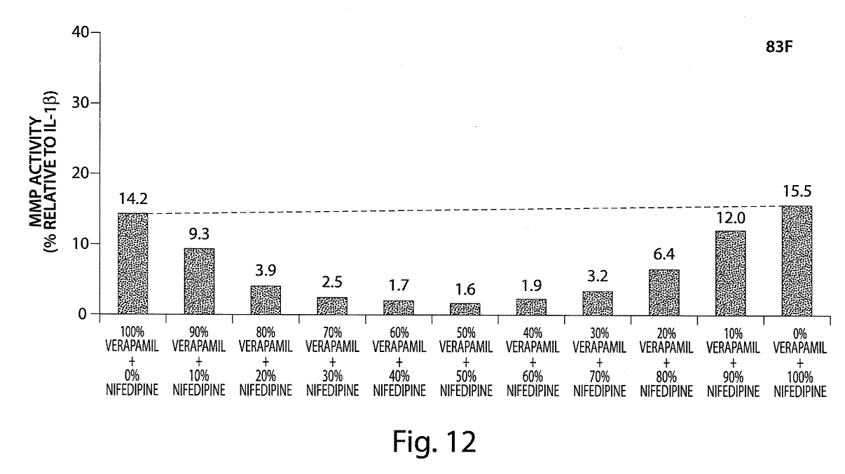

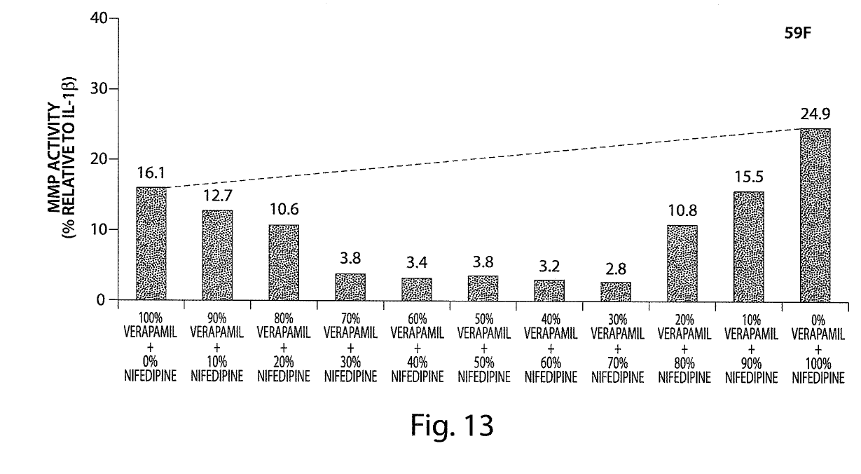

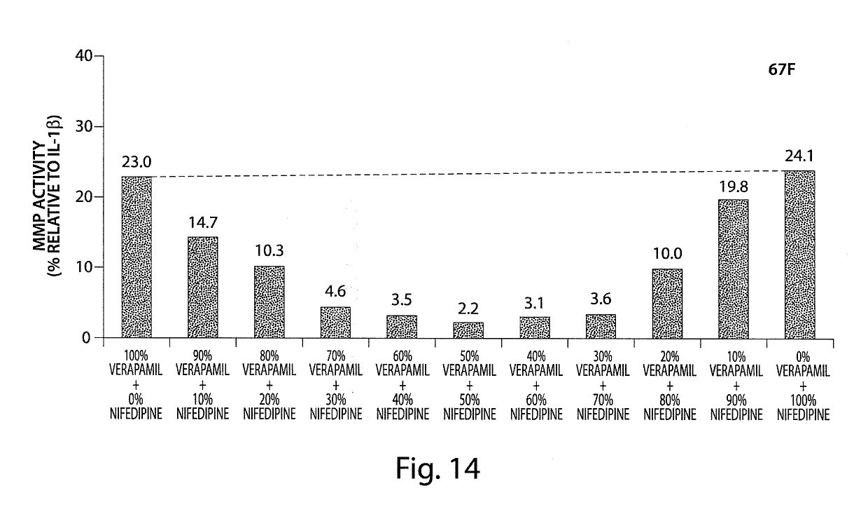

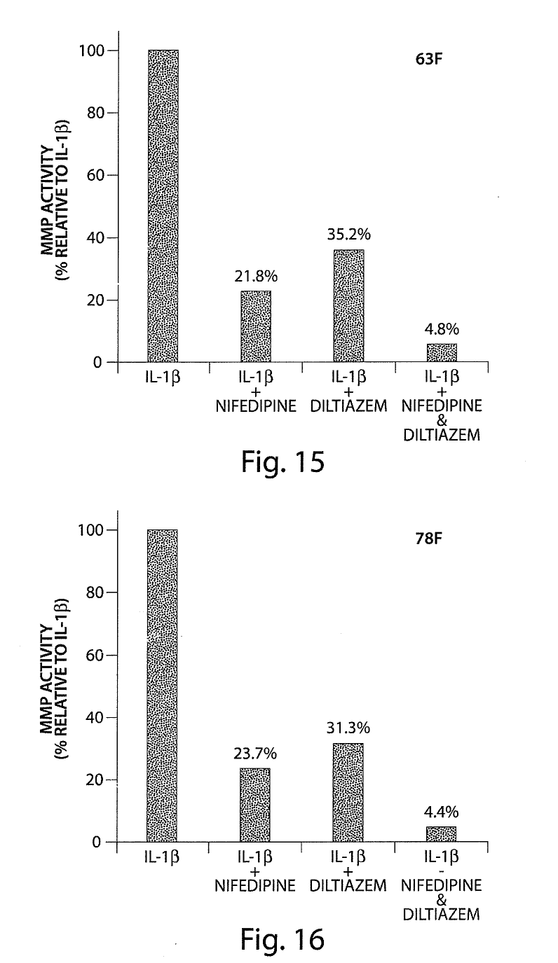

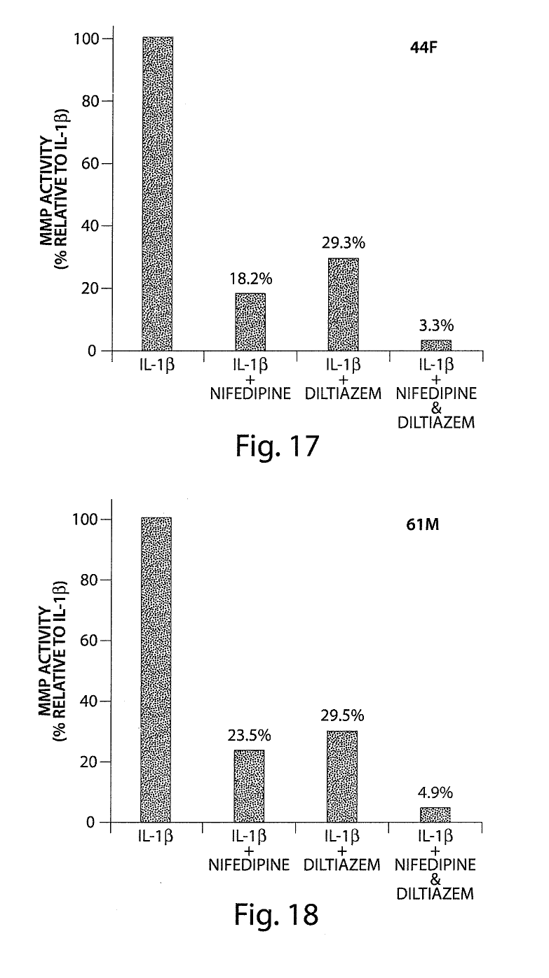

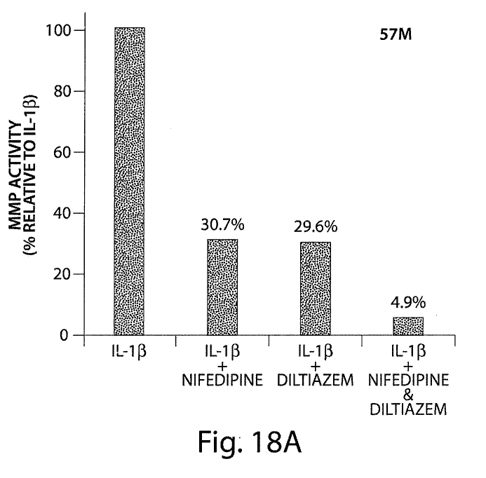

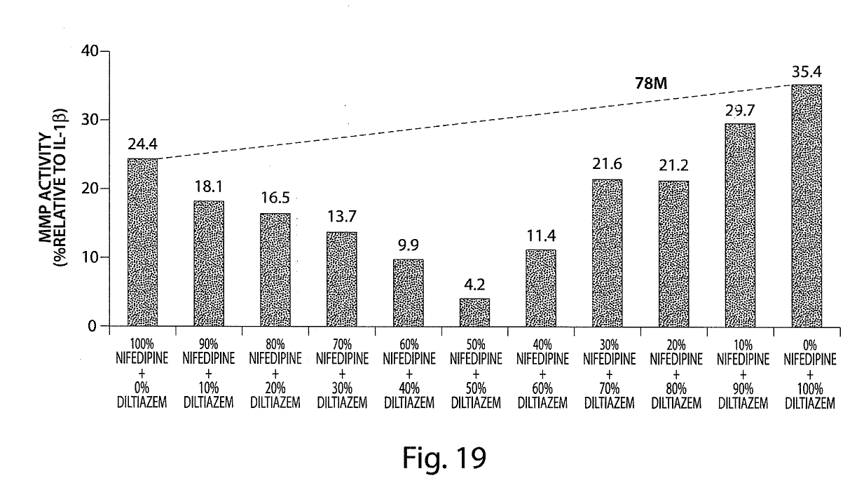

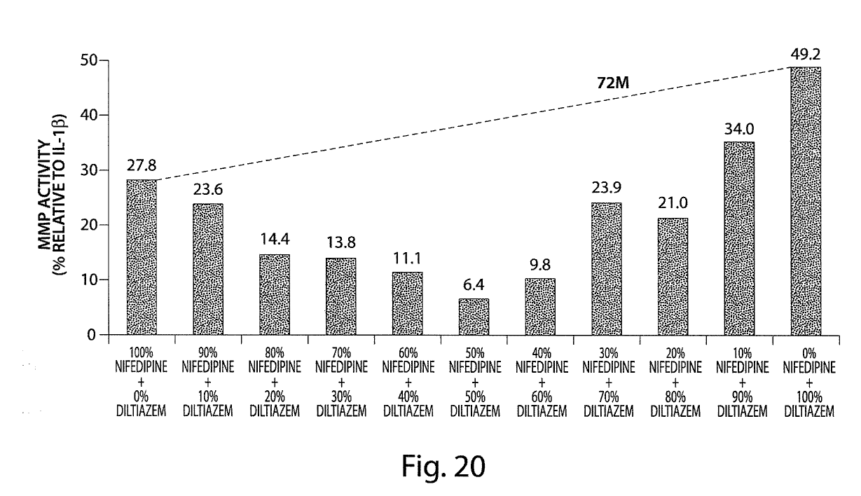

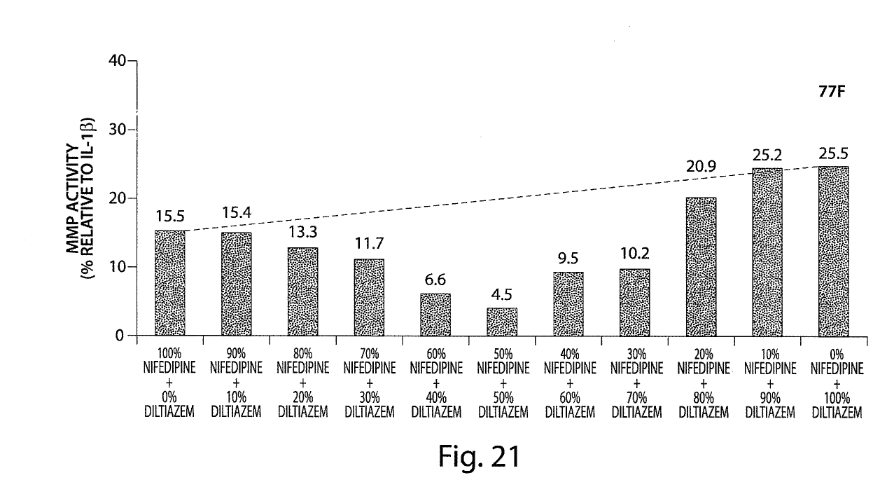

Experiments similar to those described above were conducted using compositions comprising a combination of the calcium channel regulator verapamil and the calcium-channel regulator nifedipine. See FIGS. 8-11 for summaries of the results of Examples 8-11. Further, experiments were conducted in Examples 12-14 that were similar to those conducted in the above examples. See FIGS. 12-14 for summaries of the results of Examples 12-14. See FIGS. 15-21 for summaries of the results of Examples 15-21.

### EXAMPLES 22-25

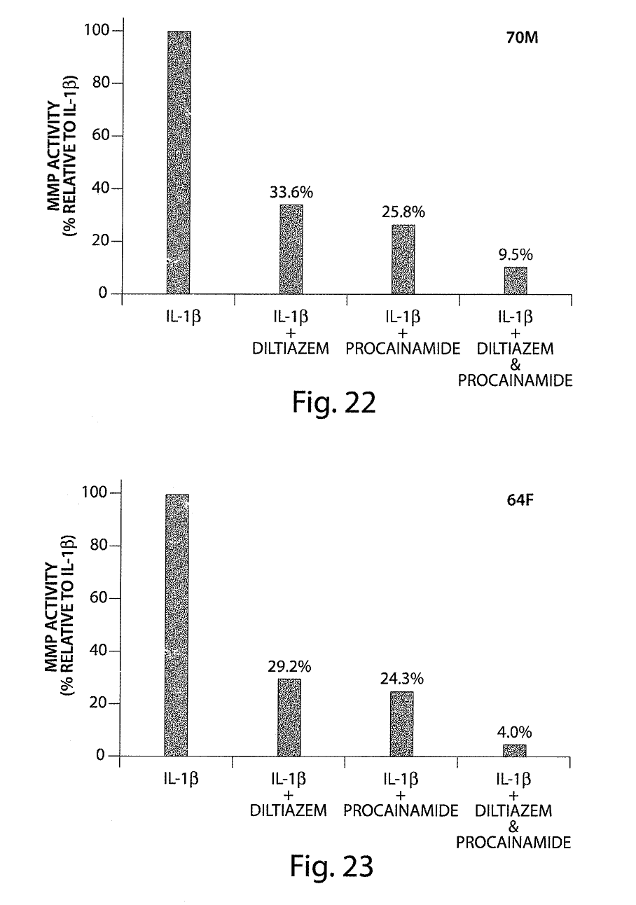

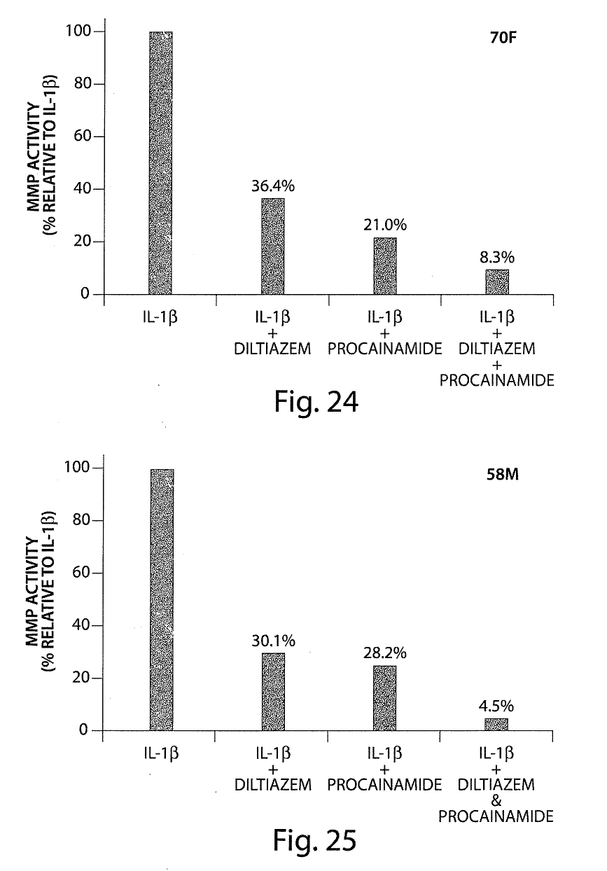

Experiments similar to those described above were conducted using compositions comprising a combination of the calcium channel regulator diltiazem and the sodium channel regulator procainamide, except that the concentration of diltiazem was 3 mg/mL and that of procainamide was 10 mg/mL. As with the previous ion-channel regulator combinations, it was found in Examples 22-25 that a combination of diltiazem and procainamide was more effective at inhibiting MMP production than either regulator alone. (See FIGS. 22-25).

### EXAMPLE 26

Study of combination of ion channel regulators at the electrophysiological level

Dose-response curves of the individual calcium-channel blockers, were determined, the EC₅₀ were computed, and the inhibition produced by specific combinations of the blockers was determined as described. A total of six concentrations of each blocker were considered: 0, EC₅₀, 4, and 16 times the EC₅₀, and ¼ and 1/16 of the EC₅₀, resulting in a total of 6×6=36 conditions.

Nystatin perforated-patch voltage-clamp whole-cell configuration was used to study the amplitude and kinetic integral characteristics of Ca currents in HIG-82 cells. Ba²⁺ ions were used as current carrier instead of Ca²⁺ because Ba²⁺ has greater conductance. The bath solution was 80 mM BaCl₂, 0.3 μM TTX, 30 mM TEA-Cl, 5 mM HEPES, pH 7.4. The electrode solution was 140 mM K-aspartate, 0.3 μM TTX, 5 mM EGTA, 10 mM HEPES, pH 7.4. The cell culture was performed as in Borisy, AA et al., PNAS 100:7977-7982.

Two standard reference models of additivism to identify synergy were used. The highest single agent (HSA) model is the larger of the effects produced by each of the combination's single agent at the same concentration as the mixture. The Bliss additivism model predicts that the combined response (C) for two single agents with effects A and B is: C=A+B−A*B. The difference in calcium current between that found in the absence of a blocker and the lowest calcium current attainable with a single blocker was considered to be 100%

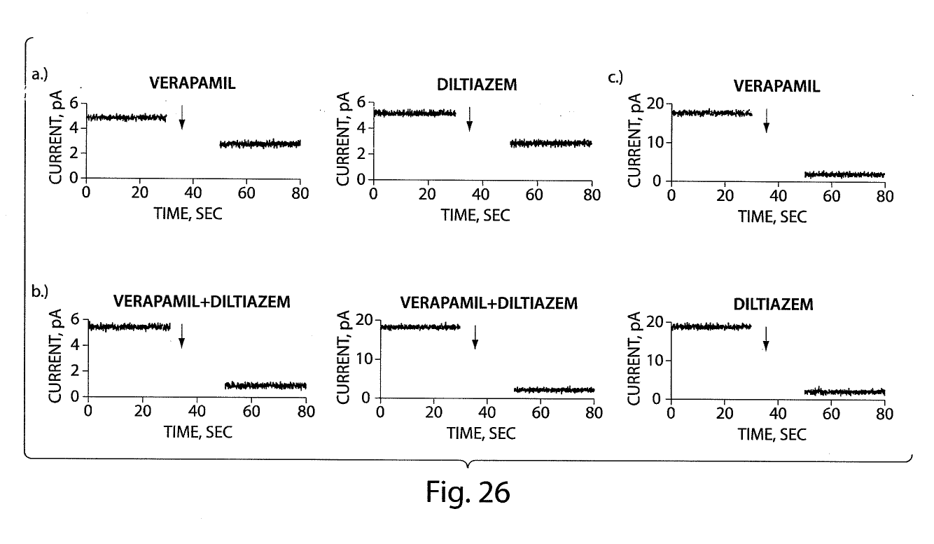

Typical results are shown in FIG. 26, which were recorded at a membrane potential of −50 mV (FIG. 26a), and 0 mV (FIG. 26bb). The traces show the effect of replacing the bath solution with one containing 100 μM verapamil, 100 μM diltiazem, or a mixture of 50 μM verapamil and 50 μM diltiazem. At −50 mV neither verapamil nor diltiazem could block the current completely. When added together, however, they blocked current almost completely, indicating that they had a synergetic effect. This effect was observed at −50 mV because the probability of the channel to be in the open state was low, thereby restricting direct access of the blockers to their binding sites from outside the cell. Access to the binding sites was increased at a membrane potential of 0 mV because the probability of the opened state was greater.

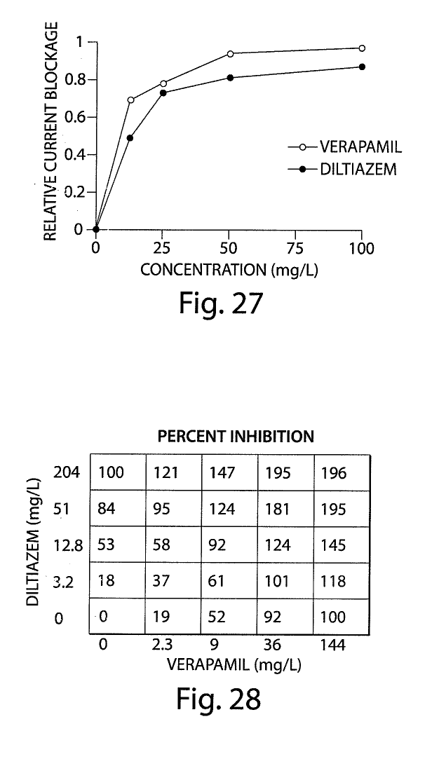

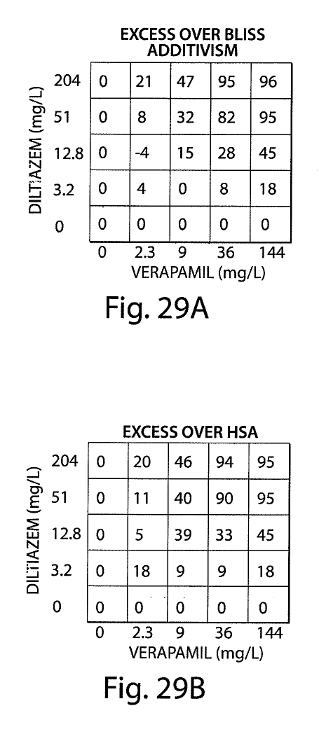

The dose-response curves are shown in FIG. 27. From these curves we determined that the EC₅₀ for verapamil was 9 mg/L, and that for diltiazem was 12.8 mg/L. The inhibition produced by verapamil and diltiazem, and their combinations is shown in FIG. 28. The combinations involving 1/16 of the EC₅₀ of the blockers were not studied because their effect on the calcium current was below the noise level of the assay (FIG. 29).

The synergistic effect of the combination of blockers in a concentration range pertinent to clinical studies is shown in FIG. 29. At low voltage 0 mV the channel fluctuated between closed and opened states with approximately equal probability of both states. Therefore verapamil and diltiazem had good excess to the binding sites, and may indicate why both substances blocked the current almost completely 0 mV, even when they were added separately. Synovial cells have a membrane potential of about −50 mV. Therefore the synergetic effect of the blockers at −50 mV (FIG. 29) may explain the synergetic effect of the blockers on MMP production by the cells.

### EXAMPLE 27

Collagen Loss Test

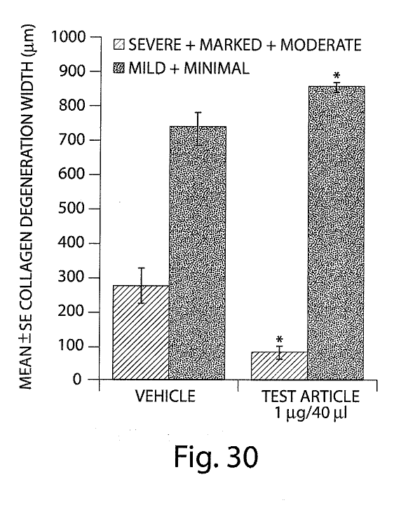

FIG. 30 depicts the results of a collagen loss test in a menisectomy model of osteoarthritis in rats, with n=15/group; and the * indicating p≦0.05 t-test to vehicle. Evaluation of meniscal tear induce osteoarthritis in rat knees requires division of the tibial plateau into 3 zones of equal widths using an ocular micrometer. The width of total cartilage degeneration is a micrometer measurement taken from the outer edge of the tibial plateau, adjacent to the osteophyte, to the point at which the cartilage is normal and represents the total extent of the tibial plateau affected by any type of degeneration.

The left hand bar of FIG. 30 represent results after administration of a placebo (saline). The right hand bars represent administration of 1 μg each of verapamil and diltiazem. The y-axis depicts the width of a tibial face showing severe/marked/moderate collagen loss in μmeters and width showing minimal or mild loss. Rats receiving 1 μg of each of verapamil and diltiazem showed significantly less severe/marked/moderate loss and more mild/minimal loss.

The foregoing description is considered as illustrative only of the principles of the invention. Further, since numerous modifications and changes will readily occur to those skilled in the art, it is not desired to limit the invention to the exact details shown and described herein, and accordingly, all suitable modifications and equivalents may be resorted to, falling within the scope of the invention. Thus, it will be apparent to those skilled in the art that many changes and substitutions can be made to the preferred embodiments herein described without departing from the spirit and scope of the present invention as defined by the appended claims.

1. A composition suitable for intra-articular injection comprising:
a first ion channel regulator and a second ion channel regulator different from the first ion channel regulator, wherein the weight ratio of the first ion channel regulator to the second ion channel regulator is about 2:8 to about 8:2; and
a pharmaceutically acceptable carrier.

2. The composition of claim 1, wherein the weight ratio of the first ion channel regulator to the second ion channel regulator is about 3:7 to about 7:3.

3. The composition of claim 1, wherein the weight ratio of the first ion channel regulator to the second ion channel regulator is about 4:6 to about 6:4.

4. The composition of claim 1, wherein the weight ratio of the first ion channel regulator to the second ion channel regulator is about 1:1.

5. The composition of claim 1, wherein the composition is substantially liquid at room temperature.

6. The composition of claim 5, wherein the concentration of the combination of the first ion channel regulator and the second channel regulator is about 0.001 mg/ml to about 2 mg/ml.

7. The composition of claim 1, wherein the first ion channel regulator and the second ion channel regulator are each independently selected from calcium channel regulators or sodium channel regulators.

8. The composition of claim 7, wherein the first ion channel regulator is chosen from verapamil, amlodipine, diltiazem, bepridil, felodipine, gallopamil, isradipine, nicardipine, nimodipine, and nitrendipine, and the second ion channel regulator is chosen from nifedipine, procainamide, quinidine, encainide, mexitil, disopyramide, and tetrodotoxin.

9. The composition of claim 7, wherein the first ion channel regulator is chosen from nifedipine, amlodipine, diltiazem, bepridil, felodipine, gallopamil, isradipine, nicardipine, nimodipine, and nitrendipine, and the second ion channel regulator is chosen from verapamil, procainamide, quinidine, encainide, mexitil, disopyramide, and tetrodotoxin.

10. The composition of claim 7, wherein the first ion channel regulator is chosen from verapamil, amlodipine, nifedipine, bepridil, felodipine, gallopamil, isradipine, nicardipine, nimodipine, and nitrendipine, and the second ion channel regulator is chosen from diltiazem, procainamide, quinidine, encainide, mexitil, disopyramide, and tetrodotoxin.

11. The composition of claim 7, wherein the first ion channel regulator and the second ion channel regulator are selected from the group consisting of verapamil, diltiazem, and nifedipine.

12. The composition of claim 1, further comprising a third ion channel regulator different from the first and second ion channel regulators.

13. The composition of claim 1, wherein the first and second ion channel regulators are a synergistic combination for regulating matrix metalloproteinases in a joint of a patient.

14. The composition of claim 1, wherein upon administration to a patient suffering from a degenerative joint disease, the composition is more effective than administering a composition that consists essentially of one ion channel regulator.

15. The composition of claim 1, wherein the pharmaceutically acceptable carrier comprises an aqueous solution.

16. The composition of claim 1, wherein the pharmaceutically acceptable carrier comprises a polymer.

17. The composition of claim 1, wherein the polymer is polyethylene glycol.

18. The composition of claim 15, wherein the pharmaceutically acceptable carrier further comprises polyethylene glycol and glycerin.

19. A method for treating a degenerative joint disease comprising administering a synergistically effective amount of a first ion channel regulator and a second ion channel regulator effective to treat the joint disease.

20. The method of claim 19, wherein the first ion channel regulator and the second ion channel regulator are administered substantially simultaneously.

21. The method of claim 19, wherein the first ion channel regulator and the second ion channel regulator are administered sequentially.

22. The method of claim 19, wherein the degenerative disease is osteoarthritis.

23. The method of claim 19, wherein the first and second ion channel regulator are administered intra-articularly by injection.

24. The method of claim 19, further comprising administering an effective amount of another degenerative joint disease treatment agent.

25. The method of claim 24, wherein the another degenerative joint disease treatment agent is selected from the group consisting of viscosupplements, corticosteroids, non-steroidal anti-inflammatory agents, analgesics, glucosamines, chondroitin, and antibiotics.

26. A method of protecting against collagen loss in a joint of a patient at risk of developing collagen loss, comprising administering a first ion channel regulator and a second ion channel regulator different from the first ion channel regulator.

27. A method of reducing collagen loss in a joint of a patient in need thereof, comprising administering intra-articularly a first ion channel regulator and a second ion channel regulator different from the first ion channel regulator, wherein the collagen loss of the patient after said administration is substantially less as compared to a patient not administered an ion channel regulator intra-articularly.

28. A kit for use in treating osteoarthritis comprising:
a container wherein a first ion channel regulator and a second ion channel regulator different than the first ion channel regulator are disposed in said container, or first ion channel regulator disposed in a first container and a second ion channel regulator disposed in a second container; and
instructions for use.

29. The kit of claim 28, wherein the container is a vial.

30. The kit of claim 28, wherein the container is a syringe.
